SEQUENCE LISTING (6 sequences)

SEQ ID NO: 1 - protein, 1255 aa, Homo sapiens [sequence omitted]

SEQ ID NO: 2 - DNA, 3768 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 3 - DNA, 200 bp, Homo sapiens
tgactgtctcctcccaaatttgtagaccctcttaagatcatgcttttcagatacttcaaagattccagaagatatgccccgggggtcctggaagccacaaggtaaacacaacacatccccctccttgactatcaattttactagaggatgtggtgggaaaaccattatttgatattaaaacaaataggcttgggatggag

SEQ ID NO: 4 - DNA, 200 bp, Homo sapiens
aaaaaaaaaagtcctttcgatgtgactgtctcctcccaaatttgtagaccctcttaagatcatgcttttcagatacttcaaagattccagaagatatgccccgggggtcctggaagccacaaggtaaacacaacacatccccctccttgactatcaattttactagaggatgtggtgggaaaaccattatttgatattaa

SEQ ID NO: 5 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 6 - DNA, 3207 bp, Homo sapiens
atgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactga

(19) United States
(12) Patent Application Publication
(10) Pub. No.: US 2020/0370102 A1
(43) Pub. Date: Nov. 26, 2020

(54) BIOMARKER INDICATING RESPONSE TO POZIOTINIB THERAPY FOR CANCER

(71) Applicants: HANMI PHARM. CO., LTD., Hwaseong-si, Gyeonggi-do (KR); NATIONAL CANCER CENTER, Goyang-si, Gyeonggi-do (KR); SAMSUNG LIFE PUBLIC WELFARE FOUNDATION, Seoul (KR)

(72) Inventors: Yeon Hee PARK, Seoul (KR); Kyung Hee PARK, Seoul (KR); Eun Jin LEE, Seongnam-si (KR); Woong Yang PARK, Seongnam-si (KR); Min Chae KIM, Seoul (KR); Young Whan PARK, Goyang-si (KR); Jung Yong KIM, Goyang-si (KR)

(73) Assignees: HANMI PHARM. CO., LTD., Hwaseong-si, Gyeonggi-do (KR); NATIONAL CANCER CENTER, Goyang-si, Gyeonggi-do (KR); SAMSUNG LIFE PUBLIC WELFARE FOUNDATION, Seoul (KR)

(21) Appl. No.: 16/763,714

(22) PCT Filed: Nov. 14, 2018
(86) PCT No.: PCT/KR2018/013882

§ 371 Date: May 13, 2020

(30) Foreign Application Priority Data
Nov. 14, 2017 (KR) 10-2017-0151831

(51) Int. Cl. C12Q 1/6827; A61K 31/517; C12Q 1/6841; A61P 35/00

(52) U.S. Cl. CPC C12Q 1/6827; A61K 31/517; C12Q 1/6841; A61K 45/06; C12Q 2600/106; C12Q 2600/156; A61P 35/00

(57) ABSTRACT

Provided are biomarkers that are sensitive or resistant to poziotinib therapy for cancer and methods of using the biomarkers.

### CROSS-REFERENCE TO RELATED APPLICATION

This application claims the benefit of Korean Patent Application No. 10-2017-0151831, filed on Nov. 14, 2017, in the Korean Intellectual Property Office, the disclosure of which is incorporated herein in its entirety by reference.

### BACKGROUND

### 1. Field

One or more embodiments relate to a method of identifying a subject having poziotinib-sensitive cancer, a method of treating a poziotinib-sensitive cancer of a subject, and a pharmaceutical composition and use for the treatment of cancer of a subject.

### 2. Description of the Related Art

Poziotinib is a low-molecular-weight compound that selectively and irreversibly inhibits the EGFR family including Her1, Her2, and Her4, and a pan-Her inhibitor having excellent inhibitory effects on the activation of EGFR and Her2 and resistant mutants. Poziotinib inhibits the growth of cancer cells of various carcinomas, the cancer cells having overexpression of Her1 or Her2 in vitro or activated mutations. In addition, poziotinib effectively blocks tumor growth in a xenotransplantation animal model having a body into which such a tumor cell has been transplanted.

However, it is not known how to identify sensitive or resistant cells by using biomarkers that exhibit sensitivity or resistance to poziotinib.

### SUMMARY

One or more embodiments include a method of identifying a subject having poziotinib-sensitive cancer.

One or more embodiments include a method of treating poziotinib-sensitive cancer in a subject.

One or more embodiments include a pharmaceutical composition for the treatment of cancer of a subject.

One or more embodiments include use of poziotinib in the preparation of medicaments for the treatment of cancer.

One or more embodiments include a method of identifying a subject having poziotinib-resistant cancer.

One or more embodiments include a method of treating poziotinib-resistant cancer in a subject.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram showing the association of the mutation with the prognosis (A) and PFS (B) regarding the ERBB3 gene (mutation number=10);

FIG. 2 is a diagram showing the association of the mutation with the prognosis (A, B) and PFS (C) regarding the BARD1 gene (mutation number=9);

FIG. 3 is a diagram showing the association of the mutation with the prognosis (A) and PFS (B) regarding the NOTCH3 gene (mutation number=13);

FIG. 4 is a diagram showing the association of the mutation with the prognosis (A, B) and PFS (B) regarding the SH2B3 gene (mutation number=6);

FIG. 5 is a diagram showing the association of the mutation with the prognosis (A) and PFS (B) regarding the SETBP1 gene (mutation number=13);

FIG. 6 is a diagram showing the association of the mutation with the prognosis (A) and PFS (B) regarding the PIK3CA gene (mutation number=34);

FIG. 7 is a diagram showing the association of the gene amplification with the prognosis (A, B, and C) and PFS (D) regarding the amplification of CDK12 gene (number of amplified subjects=42);

FIG. 8 is a diagram showing the association of the gene deletion with the prognosis (A) and PFS (B) regarding BRCA1 gene deletion (deleted subject number=⁹);

FIG. 9 is a diagram showing the association of the gene deletion with the prognosis (A) and PFS (B) regarding STAT3 gene deletion (deleted subject number=5);

FIG. 10 is a diagram showing the association of the gene copy number variation and the prognosis (A) and PFS (B) with respect to FGFR3 gene deletion or amplification (mutation number=5);

FIG. 11 illustrates tables showing the correlation between ERBB2 mutation and prognosis;

FIG. 12 illustrates graphs showing the correlation between ERBB2 mutation and PFS;

FIG. 13 shows genotype analysis in the ERBB2 gene and upstream region thereof and prognosis following the treatment with poziotinib, regarding 13 patients with mutations from among 75 patients;

FIG. 14 shows genotype analysis in the PIK3CA gene and prognosis following the treatment with poziotinib, regarding 34 patients with mutations from among 75 patients;

FIG. 15 shows genotype analysis in the ERBB3 gene and prognosis following the treatment with poziotinib, regarding 10 patients with mutations from among 75 patients;

FIG. 16 shows genotype analysis in the BARD1 gene and prognosis following the treatment with poziotinib, regarding 9 patients with mutations from among 75 patients;

FIG. 17 shows genotype analysis in the NOTCH3 gene and prognosis following the treatment with poziotinib, regarding 12 patients with mutations from among 75 patients;

FIG. 18 shows genotype analysis in the SH2B3 gene and prognosis following the treatment with poziotinib, regarding 6 patients with mutations from among 75 patients; and

FIG. 19 shows genotype analysis in the SETBP1 gene and prognosis following the treatment with poziotinib, regarding 13 patients with mutations from among 75 patients.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list).

The term “subject” used herein refers to any individual or patient on which the present disclosure is applied or performed. The subject may be an animal including a human. The animal may be a mammal. The animal includes rodents including mice, rats, hamsters and guinea pigs; cats, dogs, rabbits, cows, horses, goats, sheep, pigs, and primates (monkeys, chimpanzees, orangutans and gorillas).

The term “sensitive to poziotinib” or “poziotinib-sensitive cancer” used herein indicates cells or cancer having a slower growth when poziotinib exists than when poziotinib does not exist. The term “sensitivity” may indicate the cytotoxic or cytostatic effects of poziotinib with respect to cells. Sensitive cells or cell lines may have, in the presence of poziotinib, a growth rate that is changed 1.5, 2, 3, 4, 5, 6, 7, 8, 9, 10, 15, 20, 25 or more times. Sensitivity may be measured by a change in genome sequence or gene copy number, or an increase or decrease in expression of specific protein or mRNA. Regarding a sensitive cell or cell line, the expression of specific protein or mRNA may be changed 1.5, 2, 3, 4, 5, 6, 7, 8, 9, 10, 15, 20, or 25 or more times.

The term “resistant to poziotinib” or “poziotinib-resistant cancer” used herein refers to cells or cancer which have a normal (or basal) growth in the presence of poziotinib, and even in the absence of poziotinib, a growth that is similar to the growth level in the presence of poziotinib. Resistance may be measured by, in the presence of poziotinib, relative maintenance of cell growth rate or a change in the genome sequence or the copy number of gene, or an increase or decrease in expression of specific protein or expression of mRNA.

Regarding resistant cells or cell lines, in the presence of poziotinib, one or more resistance biomarker parameters may be changed 1.5, 2, 3, 4, 5, 6, 7, 8, 9, 10, 15, 20, 25, or more times.

The term “therapeutically effective amount” or “effective amount” used herein refers to an amount of poziotinib that is enough to improve symptoms, for example, enough to treat, heal, prevent or improve the related medical states, or enough to cause an increase in the rate of the treatment, healing, prevention, or improvement of conditions, or that provides a statistically significant improvement in the group of typically treated patients. When referring to an individual active ingredient that is administered alone, a therapeutically effective amount or dose refers only to that ingredient. When referring to a combination, a therapeutically effective amount or dose refers the combined amount of an active ingredient that results in a therapeutic effect, regardless of how it is administered in combination, including serially or simultaneously. In various embodiments, the therapeutically effective amount of poziotinib may lead to improvement in symptoms associated with cancer, including appetite, oral pain, epigastric pain, fatigue, abdominal swelling, persistent pain, bone pain, nausea, vomiting, constipation, weight loss, headache, rectal bleeding, [symbol] dyspepsia, and pain urination.

The term “treatment” used herein refers to prophylactic treatment or therapeutic treatment. In one or more embodiments, “treatment” refers to administering a compound or composition to a subject for therapeutic or prophylactic purposes.

The term “therapeutic” treatment used herein refers to administration to a subject exhibiting pathological signs or symptoms to reduce or eliminate signs or symptoms. The signs or symptoms may be biochemical, cellular, histological, functional or physical, or subjective or objective.

The “prophylactic” treatment used herein refers to administration to a subject who does not show signs of disease or shows only initial signs of disease to reduce the risk of pathology. The compound or composition used herein may be provided as a prophylactic treatment to reduce the likelihood of pathology, or to minimize the severity of the pathology when disease occurs.

The term “pharmaceutical composition” used herein refers to a composition suitable for pharmaceutical use in humans and an animal including mammals. The pharmaceutical composition may include a therapeutically effective amount of poziotinib used herein or other product, optionally, other biologically active agents, and optionally a pharmaceutically acceptable excipient, a pharmaceutically acceptable carrier, or a pharmaceutically acceptable diluent. In one embodiment, the pharmaceutical composition may include, in addition to a composition including an active ingredient and an inactive component constituting a carrier, a product that is generated directly or indirectly from combination, complexation or aggregation of one or more components, dissociation of one or more components, or other types of reaction or interaction of one or more components. Accordingly, a pharmaceutical composition according to the present disclosure includes any composition prepared by mixing a compound according to the present disclosure with an excipient, a carrier or a diluent, wherein the excipient, the carrier, and the diluent are pharmaceutically acceptable. The pharmaceutical composition may be a unit dosage form. The pharmaceutical composition may have an oral or parenteral formulation. The pharmaceutical composition may be in the form of tablets or injections. An example of a pharmaceutical composition including poziotinib is disclosed in U.S. Patent Publication No. US20130071452A1, the content of which is incorporated herein by reference.

The term “pharmaceutically acceptable carrier” used herein refers to any standard pharmaceutical carrier, such as a phosphate buffered saline solution, 5% aqueous solution and emulsion of dextrose (for example, oil/water or water/oil emulsion), buffer, or the like. Non-limiting examples of the excipient include adjuvants, binders, fillers, diluents, disintegrants, emulsifiers, wetting agents, lubricants, sweeteners, fragrances, and colorants. The pharmaceutical carrier depends on the intended method of administration of the active agent. An administration method of the related art includes an intestinal (for example, oral) administration or a parenteral (for example, subcutaneous, intramuscular, intravenous or intraperitoneal injection, or topical, transdermal or mucosal) administration.

A “pharmaceutically acceptable” or “pharmacologically acceptable” salt of the active agent used herein refers to a substance that is suitable in biological aspects or otherwise. That is, the salt may be administered to a subject without causing undesirable biological effects or harmfully interacting with any component of a composition including the salt or with any component that exists on or in the subject.

The term “nucleic acid” or “oligonucleotide” or “polynucleotide” or a grammatical equivalent thereof used herein refers to two or more nucleotides covalently linked together. Nucleic acids are generally single-stranded or double-stranded deoxyribonucleotides or ribonucleotide polymers (either pure or mixed). This term may include a nucleic acid containing naturally occurring and non-naturally occurring nucleotide analogs or modified backbone residues or linkage, the nucleic acid having a linkage, structure, or functional properties, all similar to a reference nucleic acid, and metabolized in a manner similar to reference nucleotides. Non-limiting examples of such analogs include phosphorothioate, phosphoramidate, methylphosphonate, chiral-methylphosphonate, 2-O-methyl ribonucleotide, and peptide-nucleic acid (PNA). In some cases, the nucleic acid may include nucleic acid analogs having one or more different linkages, for example, phosphoramidate linkage, phosphorothioate linkage, phosphorodithioate linkage, or O-methyl phosphoramidite linkage. In one embodiment, the nucleic acid may include a phosphodiester ester linkage. The term nucleic acid may, in some circumstances, be used interchangeably with genes, cDNA, mRNA, oligonucleotides, and polynucleotides.

The term “polypeptide”, “peptide”, and “protein” are used interchangeably to refer to polymers of amino acid residues. Amino acids may be represented by the commonly known three letter symbol or by a single letter symbol as recommended by the IUPAC-IUB Biochemical Nomenclature Commission.

The terms “sample” and “biological sample” refer to any sample suitable for the present disclosure herein. The sample contains a nucleic acid and/or a protein. The biological sample according to the present disclosure may be a tissue specimen, for example, a biopsy specimen selected from saliva biopsy, core needle biopsy, and excision biopsy.) In one embodiment, the biological sample according to the present disclosure may be a body fluid, for example, blood, serum, plasma, sputum, lung aspirate, or urine.

The term “amplification” used herein refers to a case in which, when used in connection with genes or amplicons, log₂(ratio)>1, that is, the amplification event results in two or more times greater than the number of the genes or amplicons compared to normal cells. Herein, regarding log₂(ratio), the ratio represents (the copy number of target cells/the copy number of normal cells). A positive log₂(ratio)(also called “positive log-ratio”) represents the gain in the copy number of DNA, and a negative log₂(ratio)(also called “negative log-ratio”) represents the loss or deletion in the copy number of DNA. Accordingly, the loss or deletion in the copy number of DNA indicates that the value (the copy number of target cells/the copy number of normal cells) is less than 1, that is, the copy number of target cells is smaller than the copy number of normal cells. The term “loss” and “deletion” in the copy number of DNA are interchangeably used. The copy number amplification of the gene (may be at least 1, at least 2, at least 3, at least 6, or at least 7 of log₂(ratio).

The term “normal cells” or “corresponding normal cells” used herein refers to the same type of cells derived from the same organ as cancer cells are originated from. In one aspect, the corresponding normal cells include a sample of cells obtained from a healthy human. Such corresponding normal cells may, but need not, be obtained from subjects that are age-matched with and/or have an identical gender to a subject providing the cancer cells being tested. In another aspect, the corresponding normal cells may include a sample of cells obtained from a portion of other healthy tissues of a subject with cancer. In one or more embodiments, the determination of genomic amplification may be made by comparing the genome of cancer to that of normal cells.

A first aspect of the present disclosure provides a pharmaceutical composition for the treatment of cancer of a subject, the pharmaceutical composition including poziotinib, wherein the subject has cancer cells selected from copy number amplification of the ERBB2 gene, one or more mutations in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation the FGFR3 gene.

The cancer may be breast cancer, ovarian cancer, head and neck cancer, lung cancer, gastric cancer, colon cancer, kidney cancer, blood cancer, or pancreatic cancer.

Poziotinib, that is, 1-[4-[4-(3,4-dichloro-2-fluoroanilino)-7-methoxyquinazolin-6-yl]oxypiperidin-1-yl]prop-2-en-1-one, or a pharmaceutically acceptable hydrate and/or salt thereof is represented by Formula 1.

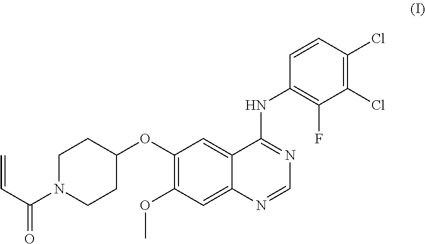 [SMILES: C=CC(=O)N1CCC(Oc2cc3c(Nc4ccc(Cl)c(Cl)c4F)ncnc3cc2OC)CC1]

The pharmaceutically acceptable salt may be an inorganic salt, an organic acid salt, or a metal salt. The inorganic salt may be a hydrochloride, a phosphate, a sulfate, or a disulfuric acid salt. The organic acid salt may be a salt of malic acid, maleic acid, citric acid, fumaric acid, besylic acid, camsylic acid, or eddylic acid. The metal salt may be a calcium salt, a sodium salt, a magnesium salt, a strontium salt, or a potassium salt. In one embodiment, poziotinib is a hydrochloride salt and may be a tablet. Poziotinib may be administered in an amount of 0.1 mg/body weight kg to 50 mg/body weight kg per day.

Poziotinib is a low molecular weight compound that selectively and irreversibly inhibits the EGFR family including Her1, Her2, and Her4, and a pan-Her inhibitor having excellent inhibitory effects on the activation of EGFR and Her2 and resistant mutants. The activity of poziotinib is disclosed in U.S. Pat. No. 8,188,102B and 2013/0071452A1, which are hereby incorporated by reference. The compound of Formula I in U.S. Pat. No. 8,188,102B is the compound of Example 36. Poziotinib inhibits the growth of cancer cells of various carcinomas, the cancer cells having overexpression of Her1 or Her2 in vitro or activated mutations, and effectively inhibits the growth of lung cancer cells resistant to gefitinib or erlotinib. In addition, poziotinib showed the effect of effectively blocking tumor growth in a xenotransplantation animal model having a body into which tumor cells have been transplanted. In addition, poziotinib has a broad and excellent inhibitory effect on EGFR and mutants thereof, and has an extensive and more effective treatment area including resistant areas of other known EGFR target antibody drugs and low molecular weight drugs. Based on these effects, improved effects on combination therapies with other drugs and resistance to various solid cancers, improved response rates over therapeutic agents of the related art, and survival time extension effects may be obtained.

Poziotinib is currently in clinical trials for cancer treatment for, for example, breast cancer. The present disclosure has found poziotinib-sensitive or resistant biomarkers based on the results of these clinical studies.

The pharmaceutical composition may include a therapeutically effective amount of other cancer therapeutic agent than poziotinib, and one or more of a pharmaceutically acceptable excipient, a pharmaceutically acceptable carrier, and a pharmaceutically acceptable diluent. The other cancer therapeutic agent may be an EGFR family inhibitor.

The term “HER2” used herein refers to a protein encoded by the ERBB2 gene in a human. The term “HER2” used herein refers to ERBB2 (human), HER2/neu, or Erbb2 (rodent).

The term “PIK3CA” used herein refers to phosphatidylinositol-4,5-bisphosphate 3-kinase, or catalytic subunit alpha. PIK3CA is a class I PI 3-kinase catalytic subunit, and also referred to as p110a protein. PIK3CA has the amino acid sequence of SEQ ID NO: 5 and may be encoded by the nucleotide sequence of SEQ ID NO: 6.

The term “BRAC1-associated RING protein 1 (BARD1)” used herein is a protein encoded by the BARD1 gene in humans. The human BARD1 protein has 777 amino acids and contains a RING finger domain, four ankyrin repeats and a two-tandem BRCT domain.

The term “SET binding protein 1 (SETBP1)” used herein is a protein encoded by the SETBP1 gene in humans. In humans, this gene is known to be located on long arm (q) 12.3, that is, 18q12.3, of chromosome 19.

The term “neurogenic locus notch homolog protein 3 (NOTCH3)” used herein is a protein encoded by the NOTCH3 gene in human. In humans, this gene is known to be located at p13.12, or 19p13.12, of chromosome 19.

The term “SH2B adapter protein 3 (SH2B3)” used herein is also known as a lymphocyte adapter protein (LNK) and is a protein encoded by the SH2B3 gene on chromosome 12 in humans.

The term “CDK12 cyclin-dependent kinase 12 (CDK12)” used herein refers to, in humans, a protein kinase encoded by the CDK12 gene. This enzyme is a member of the cyclin-dependent kinase protein family.

The term “BRCA1” used herein is a tumor suppressor protein. BRCA1 is known to be expressed on the chromosome of 17q12.31 in humans.

The term “signal transducer and activator of transcription 3 (STAT3)” used herein refers to a transcription factor that is encoded by the STAT3 gene in humans. STAT3 is known to be expressed on chromosome 17q21.2 in humans.

The term “fibroblast growth factor receptor 3 (FGFR3)” used herein is a member of the FGFR family and is a protein encoded by the FGFR3 gene in humans.

Regarding the composition, the subject may have breast cancer. The subject may have HER2-positive metastatic breast cancer. The subject may have previously undergone HER2-targeted cancer treatment, not treatment with a poziotinib. The cancer treatment may be a treatment with a HER2 targeted cancer therapeutic agent. The therapeutic agent may be an EGFR inhibitor. The EGFR inhibitor may be selected from erlotinib, gefitinib, lapatinib, canetinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, trastuzumab, margetuximab, panitumumab, matuzumab, necitumumab, pertuzumab, nimotuzumab, zalutumumab, necitumumab, cetuximab, icotinib, afatinib, and a pharmaceutically acceptable salt thereof. The therapeutic agent may be an anti-EGFR family antibody or a complex including the anti-EGFR family antibody. The anti-EGFR family antibody may be an anti-HER1 antibody, an anti-HER2 antibody, or an anti-HER4 antibody.

Regarding the composition, the cancer cells may have one or more, for example, two or more, three or more, or four or more mutations in the ERBB2 gene region.

The cancer cells may have one or more, for example, two or more, three or more, or four or more mutations in an extracellular domain-encoding region of the ERBB2 gene or a sequence upstream of the ERBB2 gene.

Regarding the composition, the average number of replication units of the ERBB2 gene in the cancer cells may be 2 or more, for example, 3 or more, 4 or more, 5 or more, 6 or more, 7 or more, 16 or more, 2 to 6, 2 to 5, 3 to 6, 3 to 5, or 4 to 6.

The cancer cells may be sensitive to poziotinib.

The mutation in the ERBB2 gene region may be a point mutation. The point mutation may be one that causes amino acid substitution, one that causes mRNA splicing, or a point mutation in the upstream region. The mutation may include a nucleotide mutation that causes substitution of at least one amino acid selected from Q568E, P601R, I628M, P885S, R143Q, R434Q, and E874K in the amino acid sequence of ERBB2 of SEQ ID NO: 1, and at least one substitution mutation selected from substitution of G at position 1898 with C in a nucleotide sequence encoding ERBB2 of SEQ ID NO: 2, substitution of A at position 100 with G in a nucleotide sequence of SEQ ID NO: 3, and substitution of C at position 100 with T in a nucleotide sequence of SEQ ID NO: 4. SEQ ID NOS: 1 and 3 are the amino acid sequence and nucleotide sequence of ERBB2, respectively, and SEQ ID NOS: 3 and 4 are the nucleotide sequences of the upstream region of the ERBB2 gene. The nucleotide mutation that causes substitution of at least one amino acid selected from Q568E, P601R, I628M, P885S, R143Q, R434Q, and E874K may be at least one substitution selected from substitution of C at position 1702 with G, substitution of C at position 1802 with G, substitution of C at position 1884 with G, substitution of C at position 2653 with T, substitution of G at position 428 with A, substitution of G at position 1301 with A, and substitution of G at position 2620 with A, in the nucleotide sequence of SEQ ID NO: 2.

A second aspect of the present disclosure provides use of poziotinib in the preparation of medicaments for the treatment of a subject having breast cancer, wherein the subject has breast cancer cells selected from copy number amplification of the ERBB2 gene, one or more mutations in an ERBB2 gene portion including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

A third aspect of the present disclosure provides a method of treating breast cancer in a subject, the method including administering a therapeutically effective amount of poziotinib to the subject having breast cancer, wherein breast cancer cells of the subject have at least one selected from copy number amplification of the ERBB2 gene, one or more mutations in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

In the method, the subject may have an HER2-positive metastatic cancer. The subject may have previously undergone the HER2-targeted cancer treatment. The cancer treatment may be a treatment with a HER2 targeted cancer therapeutic agent. The therapeutic agent may be an EGFR inhibitor. The EGFR inhibitor may be selected from erlotinib, gefitinib, lapatinib, canetinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, trastuzumab, margetuximab, panitumumab, matuzumab, necitumumab, pertuzumab, nimotuzumab, zalutumumab, necitumumab, cetuximab, icotinib, afatinib, and a pharmaceutically acceptable salt thereof. The therapeutic agent may be an anti-EGFR family antibody or a complex including the anti-EGFR family antibody. The anti-EGFR family antibody may be an anti-HER1 antibody, an anti-HER2 antibody, or an anti-HER4 antibody.

The cancer cells may have one or more, for example, two or more, three or more, or four or more mutations in the ERBB2 gene region.

The cancer cells may include one or more, for example, two or more, three or more, or four or more mutations in a region of the ERBB2 gene encoding the extracellular domain or the upstream region.

The average number of replication units of the ERBB2 gene in the cancer cells may be 2 or more, for example, 3 or more, 4 or more, 5 or more, 6 or more, 7 or more, 16 or more, 2 to 6, 2 to 5, 3 to 6, 3 to 5, or 4 to 6.

The cancer cells may be poziotinib-sensitive.

In the ERBB2 gene region, the mutation may be a point mutation. The point mutation may be one that causes amino acid substitution, one that causes mRNA splicing, or a point mutation in the upstream region. The mutation may include a nucleotide mutation that causes substitution of at least one amino acid selected from Q568E, P601R, I628M, P885S, R143Q, R434Q, and E874K in the amino acid sequence of ERBB2 of SEQ ID NO: 1, and at least one substitution mutation selected from substitution of G at position 1898 with C in a nucleotide sequence encoding ERBB2 of SEQ ID NO: 2, substitution of A at position 100 with G in a nucleotide sequence of SEQ ID NO: 3, and substitution of C at position 100 with T in a nucleotide sequence of SEQ ID NO: 4. The nucleotide mutation that causes substitution of at least one amino acid selected from Q568E, P601R, I628M, P885S, R143Q, R434Q, and E874K may be at least one substitution selected from substitution of C at position 1702 with G, substitution of C at position 1802 with G, substitution of C at position 1884 with G, substitution of C at position 2653 with T, substitution of G at position 428 with A, substitution of G at position 1301 with A, and substitution of G at position 2620 with A, in the nucleotide sequence of SEQ ID NO: 2.

The administration may be oral or parenteral administration. The parenteral administration includes subcutaneous injection, intravenous administration, intramuscular administration, intrathecal administration, intradermal administration, intraperitoneal administration, and the like.

A fourth aspect provides a method of treating poziotinib-sensitive cancer in a subject, the method including: detecting that a cancer cell-containing sample obtained from the subject has at least one selected from copy number amplification of the ERBB2 gene, one or more mutations in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene, wherein the having of at least one selected from copy number amplification of the ERBB2 gene, one or more mutations in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene indicates that the cancer cells are sensitive to poziotinib; and administering a therapeutically effective amount of poziotinib to the subject having poziotinib-sensitive cancer.

A fifth aspect provides a method of identifying a subject having poziotinib-sensitive cancer by identifying a cancer cell-containing sample obtained from the subject to have at least one selected from copy number amplification of the ERBB2 gene, one or more mutations in an ERBB2 gene portion including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene, wherein when the cancer cells are sensitive to poziotinib, it is confirmed that the cancer cell-containing sample has at least one selected from copy number amplification of the ERBB2 gene, one or more mutations in an ERBB2 gene portion including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

In the fourth and fifth aspects, the detecting may include requesting a test that provides analysis results for determining whether cancer cells isolated from the subject have at least one selected from copy number amplification of the ERBB2 gene, one or more mutations in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene. In the fourth aspect, a person who is different from a person who performs the administration may be requested to perform the test.

In the fourth and fifth aspects, the detecting may include providing the cancer cell-containing sample obtained from the subject. The detecting may include analyzing a nucleic acid or an expression product thereof in the sample. The analyzing may be measuring the level of mutation and the gene copy number in at least one selected from ERBB2 gene, an ERBB2 gene region including ERBB2 gene and a sequence within 10 kb upstream thereof, ERBB3 gene, BARD1 gene, SETBP1 gene, PIK3CA gene, NOTCH3 gene, SH2B3 gene, CDK12 gene, BRCA1 gene, STAT3 gene, and FGFR3 gene. The analysis may be performed by methods known in the art.

The detecting may include performing at least one analysis selected from sequencing, size analysis, primer extension analysis, allele-specific primer extension analysis, allele-specific nucleotide hybridization analysis, 5′-nuclease degradation assay, an assay using molecular beacons, single-stranded conformation polymorphism, hybridization analysis, and oligonucleotide ligation analysis. Due to these analyses, the level of mutation in a gene may be measurable.

The detecting may include measuring the average number of replication units of at least one gene selected from ERBB2 gene, ERBB3 gene, BARD1 gene, SETBP1 gene, PIK3CA gene, NOTCH3 gene, SH2B3 gene, CDK12 gene, BRCA1 gene, STAT3 gene, and FGFR3 gene to identify an increase in the average number of replication units. The measuring of the average number of replication units may be performed by methods known in the art. The measuring of the average number of replication units may include at least one analysis selected from single nucleotide polymorphism (SNP) arrays, comparative genomic hybridization (CGH), southern blot analysis, fluorescence in situ hybridization (FISH), and silver in situ hybridization (SISH).

In the fourth and fifth aspects, in the detecting, the existence of 2 or more, 3 or more, or 4 or more mutations in the ERBB2 gene region indicates that the cancer cells are sensitive to poziotinib.

In the fourth and fifth aspects, in the detecting, the existence of one or more, for example, two or more, three or more, or four or more mutations in the region of the ERBB2 gene encoding the extracellular domain or an upstream region thereof indicates that the cancer cells are sensitive to poziotinib.

In the fourth and fifth aspects, in the detecting, the existence of one or more mutations in the ERBB2 gene region and the average number of replication units of ERBB2 gene being 2 or more, for example, 3 or more, 4 or more, 5 or more, 6 or more, 7 or more, 16 or more, 2 to 6, 2 to 5, 3 to 6, 3 to 5, or 4 to 6 show that the cancer cells are sensitive to poziotinib.

In the fourth and fifth aspects, the method may include measuring the expression level of the gene in the sample. The expression level may be the level of mRNA or protein which is expressed by the gene. The measuring may include at least one selected from transcript expression arrays, RNA in situ hybridization, northern blot analysis, direct exon and transcript enumeration through transcript sequencing. The measuring of the level of protein may include a protein array (e.g., ELISA, and reverse phase protein assay-RPPA or western blot analysis of cell or tissue lysate or extract), immunohistochemical staining (IHC) analysis of tissue sections to identify the existence of target protein, or an antibody-based method for detecting increased protein expression of a target protein.

In the fourth and fifth aspects, the detecting may include providing polynucleotide and/or protein derived from cancer cells obtained from the subject; providing a mutation profile and expression profile of gene and protein of a test sample by contacting polynucleotide and/or protein with a microarray; and comparing the mutation profile and the expression profile of gene and protein with a profile of a control sample. The microarray may be a microarray on which a polynucleotide probe that links to a target nucleotide or a binding substance that links to a target protein is immobilized. The binding substance may be an antibody.

A sixth aspect provides a method of treating cancer of a subject, the method including selecting a subject of which cancer is to be treated by using poziotinib based on whether cancer cells isolated from the subject have at least one selected from copy number amplification of the ERBB2 gene, having one or more mutation in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene, wherein having of the at least one indicates that the cancer cells are sensitive to poziotinib; and administering a therapeutically effective amount of poziotinib to the selected subject.

In the method, the selecting is detecting that cancer cells obtained from the subject have at least one selected from copy number amplification of the ERBB2 gene, one or more mutations in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene, wherein having of the at least one indicates that the cancer cells are sensitive to poziotinib. The detecting is the same as described above.

The detecting may include requesting a test that provides analysis results for determining whether cancer cells isolated from the subject have at least one selected from copy number amplification of the ERBB2 gene, one or more mutations in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene. A person who is different from a person who performs the administration may be requested to perform the test.

The selecting may include providing the cancer cell-containing sample obtained from the subject; analyzing the cancer cell-containing sample to identify that the cancer cells have at least one selected from copy number amplification of the ERBB2 gene, having one or more mutation in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene; and determining the subject to be a subject that is to be treated by using poziotinib when the cancer cells have at least one selected from copy number amplification of the ERBB2 gene, having one or more mutation in an ERBB2 gene region including the ERBB2 gene and a sequence within 10 kb upstream thereof, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

In the selecting, when the cancer cells have one or more mutations, for example, two or more mutations in the ERBB2 gene region, the subject is selected as a subject that is to be treated by using poziotinib.

In the selecting, when the cancer cells have one or more mutations in the region encoding the extracellular domain of ERBB2 or the upstream of the ERBB2 gene in the ERBB2 gene region, the subject is selected as a subject that is to be treated by using poziotinib.

In the selecting, when the cancer cells have one or more mutations in the ERBB2 gene region and the average number of replication units of the ERBB2 gene is two or more, the subject is selected as a subject that is to be treated by using poziotinib.

In the first to sixth aspects, one or more mutations in the NOTCH3 gene may include at least one selected from R75Q, D1171V, C388Y, D1598V, E1161K, G1347R, R1175W, A198V, P2191L, D1443A, R1175W, R1761C, L1518M, R1309L, R1175W, and R572L. One or more mutations in SH2B3 gene may include at least one selected from I568T, A536T, R551W, A102S, and I568T.

A seventh aspect provides a method of treating cancer of a subject, the method including administering a therapeutically effective amount of therapeutic drugs other than poziotinib to the subject having cancer, wherein cancer cells of the subject have at least one selected from the existence of one or more mutations in the ERBB3 gene, the existence of one or more mutations in the BARD1 gene, the existence of one or more mutations in the SETBP1 gene, the existence of one or more mutations in the PIK3CA gene, and the existence of the copy number variation in the FGFR3 gene.

An eighth aspect provides a method of treating cancer of a subject, the method including selecting the subject as a subject that is to be treated by using therapeutic drugs other than poziotinib based on whether cancer cells isolated from the subject have at least one existence selected from the existence of one or more mutations in the ERBB3 gene, the existence of one or more mutations in the BARD1 gene, the existence of one or more mutations in the SETBP1 gene, the existence of one or more mutations in the PIK3CA gene, and the existence of the copy number variation in the FGFR3 gene, wherein the having of one or more existence indicates that the cancer cells are resistant to poziotinib; and administering a therapeutically effective amount of other cancer therapeutic drugs than poziotinib to the selected subject.

A ninth aspect provides a method of treating poziotinib-resistant cancer in a subject, the method including detecting that a cancer cell-containing sample obtained from a subject has at least one existence selected from the existence of one or more mutations in the ERBB3 gene, the existence of one or more mutations in the BARD1 gene, the existence of one or more mutations in the SETBP1 gene, the existence of one or more mutations in the PIK3CA gene, and the existence of the copy number variation in the FGFR3 gene, wherein the having of one or more existence indicates that the cancer cells are resistant to poziotinib; and administering a therapeutically effective amount of cancer therapeutic drugs other than poziotinib to the subject.

A tenth aspect provides a method of identifying a subject having poziotinib-resistant cancer, the method including detecting that a cancer cell-containing sample obtained from the subject has at least one existence selected from the existence of one or more mutations in the ERBB3 gene, the existence of one or more mutations in the BARD1 gene, the existence of one or more mutations in the SETBP1 gene, the existence of one or more mutations in the PIK3CA gene, and the existence of the copy number variation in the FGFR3 gene, wherein the having of one or more existence indicates that the cancer cells are resistant to poziotinib.

In the seventh to tenth aspects, one or more mutations in ERBB3 gene may be a nucleotide mutation causing at least one selected from T355I, R967K, R1127H, E1189K, T1342K, R1127H, and 1093_1096 del, one or more mutations in BARD1 gene may be a nucleotide mutation causing at least one selected from 359_369 del and V571E, one or more mutations in SETBP1 gene may be a nucleotide mutation causing at least one selected from V1450M, R1008H, T1078H, H1206L, E1466D, R627C, E740K, and E1466D, and one or more mutations in PIK3CA gene may be a nucleotide mutation causing at least one selected from I889M, E542K, H1047R, H1047L, and E545K.

According to a method of identifying a subject having poziotinib-sensitive cancer, the subject having poziotinib-sensitive cancer is effectively identified.

According to a method of treating poziotinib-sensitive cancer of a subject, poziotinib-sensitive cancer is effectively treated.

A pharmaceutical composition for the treatment of cancer of a subject is used to treat poziotinib-sensitive cancer in the subject.

In the preparation of medicaments for the treatment of cancer, regarding use of poziotinib, poziotinib is effectively used in the preparation of medicaments to be administered to a subject having cancer.

According to a method of identifying a subject having poziotinib-resistant cancer, the subject having poziotinib-resistant cancer is effectively identified.

According to a method of treating poziotinib-resistant cancer of a subject, poziotinib-resistant cancer is effectively treated in a subject.

Hereinafter, the present disclosure will be described in more detail with reference to examples. However, these examples are for illustrative purposes only, and the scope of the present disclosure is not limited to these examples.

### Example 1: Efficacy of Poziotinib According to Genetic Information about Breast Cancer Patients

Poziotinib was administered to breast cancer patients and confirmed the efficacy thereof according to the genotypes of breast cancer patients.

1. Administration of Poziotinib and Genotype Analysis of Breast Cancer Patients

Poziotinib is a pan-HER inhibitor small molecule breast cancer therapeutic agent under clinical development. Poziotinib showed potent anti-tumor activity through irreversible inhibition of HER family tyrosine kinase in preclinical and early clinical studies. Recently, through an open-label, multicenter, two-phase study of poziotinib monotherapy, it was evaluated whether poziotinib was an option for breast cancer patients with HER2-positive metastatic breast cancer who twice or more experienced the failure of 2 HER2 target treatments. The genetic profile of HER2-positive metastatic breast cancer was evaluated, and the potential biomarkers of poziotinib for HER2-positive metastatic breast cancer (MBC) were investigated.

The experimental method is as follows. All participants with MBC were diagnosed with HER2-positive metastatic breast cancer according to the American Society of Clinical Oncology/College of-American Pathologist Her2 guideline. Fresh tissue samples or FFPE samples were obtained from these patients and used to extract DNA and RNA for next generation sequencing (NGS).

DNA and RNA were extracted from the samples and NGS was performed thereon. Targeted deep sequencing was performed by using a customized 381 cancer gene panel (CancerSCAN™, Samsung Hospital, Inc.) and the correlation among sequencing data, immunohistochemistry, and clinical outcome was analyzed.

An effective amount of poziotinib was administered to the breast cancer patients. The prognosis of the patients was observed. Prognosis was classified into partial remission (PR), stable disease (SD), and progressive disease (PD). For each patient, progressive free survival (PFS) was also identified.

2. Sensitivity Analysis of Genotype and Poziotinib Treatment

From April 2015 to February 2016, 106 patients enrolled for clinical trials. Biomarker data were available for 75 of these patients.

Among the 75 patients, PR was 14, SD was 41, and PD was 20. The gene has 129 mutations and a copy number variant (CNV). CNV having a frequency of mutation of 5 or more was selected. The significance threshold is a p-value<0.05. The score of HER IHC was 3+ for 63 breast cancer patients and 2+ for 12 patients. IHC score of HER2 was positively correlated with copy number (CN) of HER2 (p=0.001), but 11 breast cancer tissues showed no HER2 copy number amplification (6 patients had HER2 IHC score of 2+ and 5 patients had HER2 IHC score of 3+).

As a result, the ERBB3 gene (mutation number=10), the BARD1 gene (mutation number=9), the SETBP1 gene (mutation number=13), and the PIK3CA gene (mutation number=34) were associated with poor prognosis in the presence of mutations. NOTCH gene (mutation number=13), and SH2B3 gene (mutation number=6) were associated with good prognosis in the presence of mutations. CDK12 (the number of subjects having copy number amplification=42) and the copy number amplification of the ERBB2 gene were each associated with good prognosis. Copy number deletion of each of the BRCA1 gene and STAT3 gene were associated with bad prognosis. Deletion or amplification of the FGFR3 gene (the number of copy number variations=5) was associated with bad prognosis.

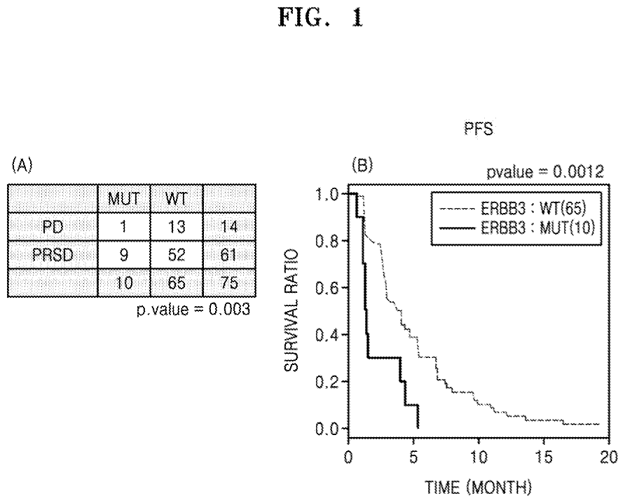

FIG. 1 is a diagram showing the association of the mutation with the prognosis (A) and PFS (B) regarding the ERBB3 gene (mutation number=10). As shown in FIG. 1, when ERBB3 gene has a mutation, the mutation was associated with prognosis and improvement of PFS (p-value of (A) was 0.003 and p-value of (B) was 0.0012).

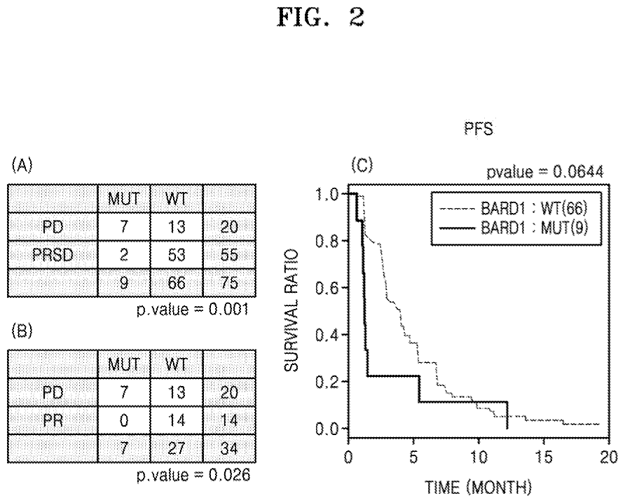

FIG. 2 is a diagram showing the association of the mutation with the prognosis (A), (B), and PFS (C) regarding the BARD1 gene (mutation number=9). As shown in FIG. 2, when BARD1 gene has a mutation, the mutation was associated with worsening of prognosis. Referring to FIGS. 2, (A) and (B) show fisher exact test results of PD (the number of subjects having progressive disease) vs (PRSD (the number of subjects having partial remission and stable disease), and PD (the number of subjects having progressive disease) vs PR (the number of subjects having partial remission) (p-value of (A) was 0.001 and p-value of (B) was 0.0026), and, (C) shows PFS.

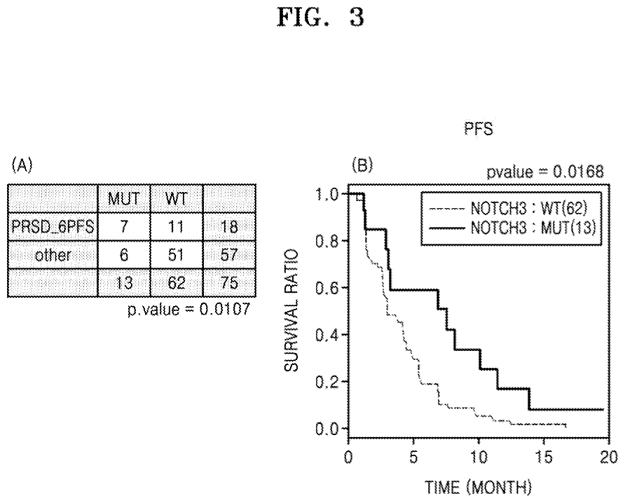

FIG. 3 is a diagram showing the association of the mutation with the prognosis (A) and PFS (B) regarding the NOTCH3 gene (mutation number=13). As shown in FIG. 3, when NOTCH3 gene has a mutation, the mutation was associated with prognosis and improvement of PFS (p-value of (A) was 0.0107 and p-value of (B) was 0.0168).

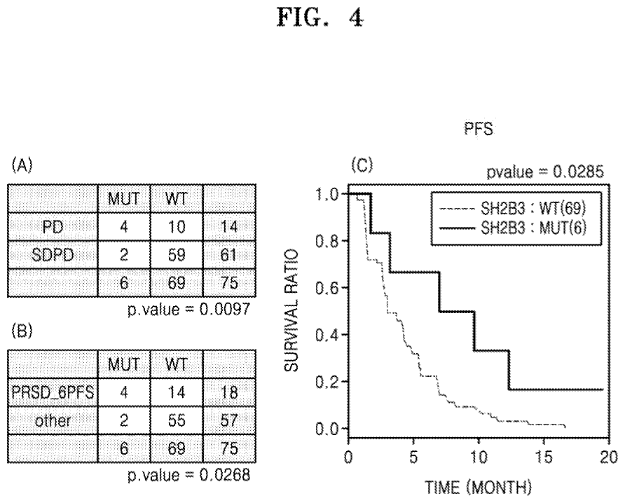

FIG. 4 is a diagram showing the association of the mutation with the prognosis (A), (B), and PFS (C) regarding the SH2B3 gene (mutation number=6). As shown in FIG. 4, when SH2B3 gene has a mutation, the mutation was associated with prognosis and improvement of PFS. P-value of (A), p-value of (B), and p-value of (C) were 0.0097, 0.0266, and 0.0285, respectively.

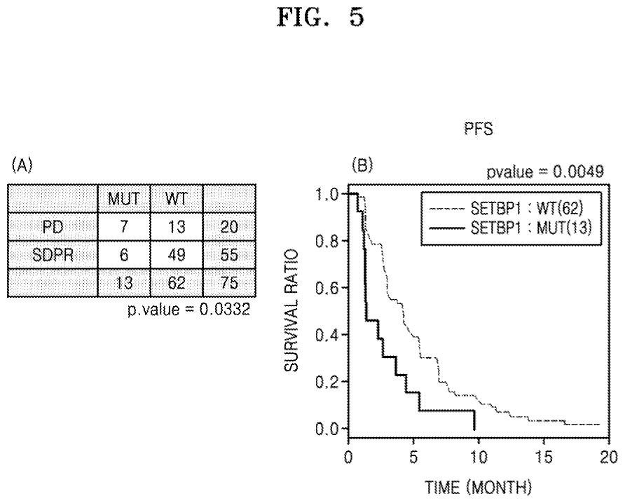

FIG. 5 is a diagram showing the association of the mutation with the prognosis (A) and PFS (B) regarding the SETBP1 gene (mutation number=13). As shown in FIG. 5, when SETBP1 gene has a mutation, the mutation was associated with prognosis and improvement of PFS (p-value of (A) was 0.0332 and p-value of (B) was 0.0049).

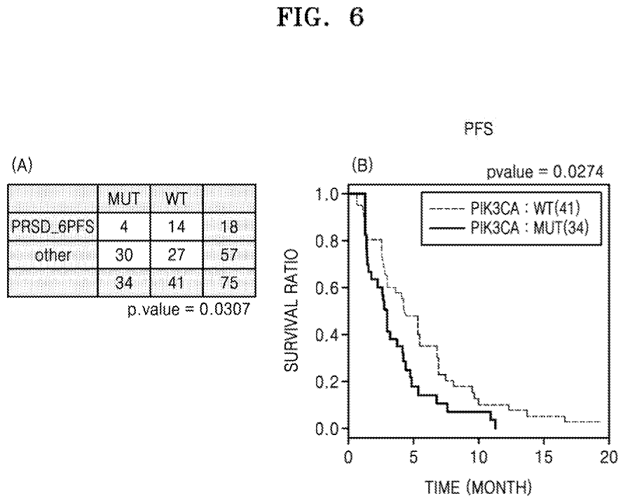

FIG. 6 is a diagram showing the association of the mutation with the prognosis (A) and PFS (B) regarding the PIK3CA gene (mutation number=34). As shown in FIG. 6, when PIK3CA gene has a mutation, the mutation was associated with prognosis and worsening of PFS (p-value of (A) was 0.0307 and p-value of (B) was 0.0274).

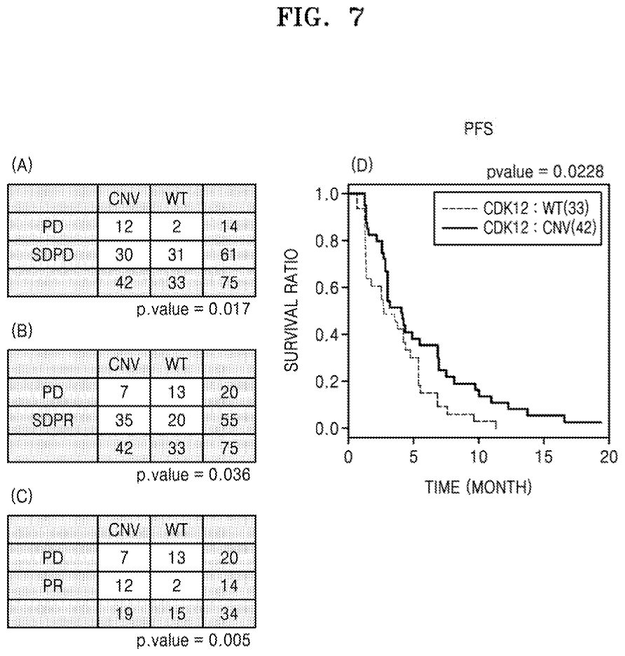

FIG. 7 is a diagram showing the association of the gene amplification with the prognosis (A, B, and C) and PFS (D) regarding the amplification of CDK12 gene (number of subjects having amplification=42). As shown in FIG. 7, when CDK12 gene has a mutation, the mutation was associated with prognosis and improvement of PFS.

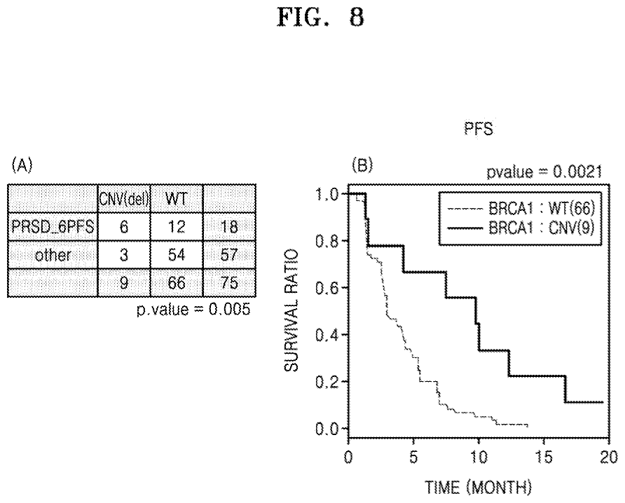

FIG. 8 is a diagram showing the association of the gene deletion with the prognosis (A) and PFS (B) regarding BRCA1 gene deletion (number of subjects having deletion=9). As shown in FIG. 8, when BRCA1 gene has a deletion, the mutation was associated with prognosis and improvement of PFS.

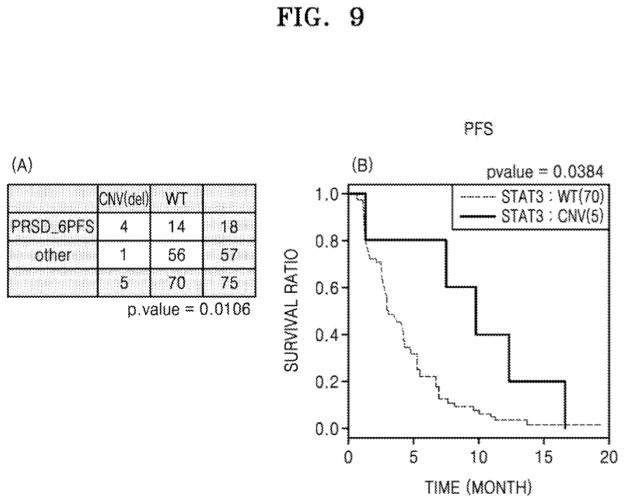

FIG. 9 is a diagram showing the association of the gene deletion with the prognosis (A) and PFS (B) regarding STAT3 gene deletion (number of subjects having deletion=5). As shown in FIG. 9, when STAT3 gene has a deletion, the mutation was associated with prognosis and improvement of PFS.

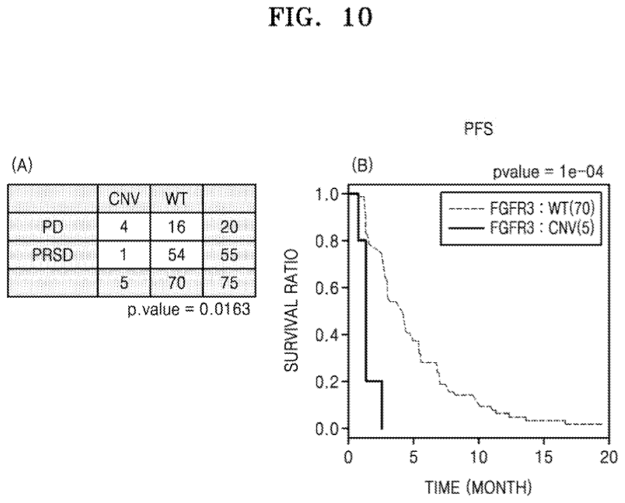

FIG. 10 is a diagram showing the association of the gene copy number variation and the prognosis (A) and PFS (B) with respect to FGFR3 gene deletion or amplification (mutation number=5). As shown in FIG. 10, when FGFR3 gene has deletion or amplification, the mutation was associated with prognosis and worsening of PFS.

Regarding ERBB2 gene, amplification analysis was performed and the relationship between mutation and symptoms and PFS was identified. Of the 75 patients, 13 patients had mutations in or upstream of the ERBB2 gene. A total number of mutations was 18, and some patients had multiple mutations. A mutation showing positive prognosis with respect to the treatment with poziotinib, that is, partial remission was mostly located in the extracellular domain or the upstream.

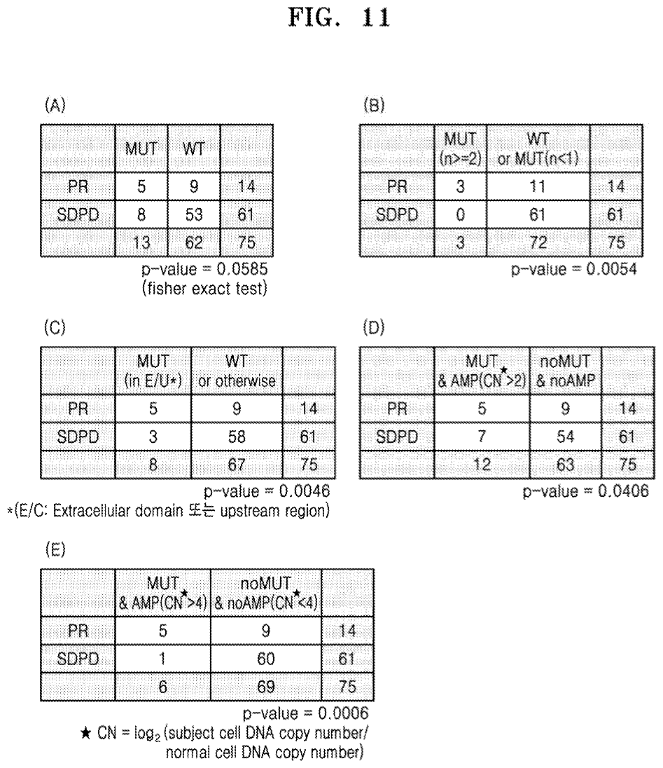

FIG. 11 illustrates tables showing the correlation between ERBB2 mutation and prognosis.

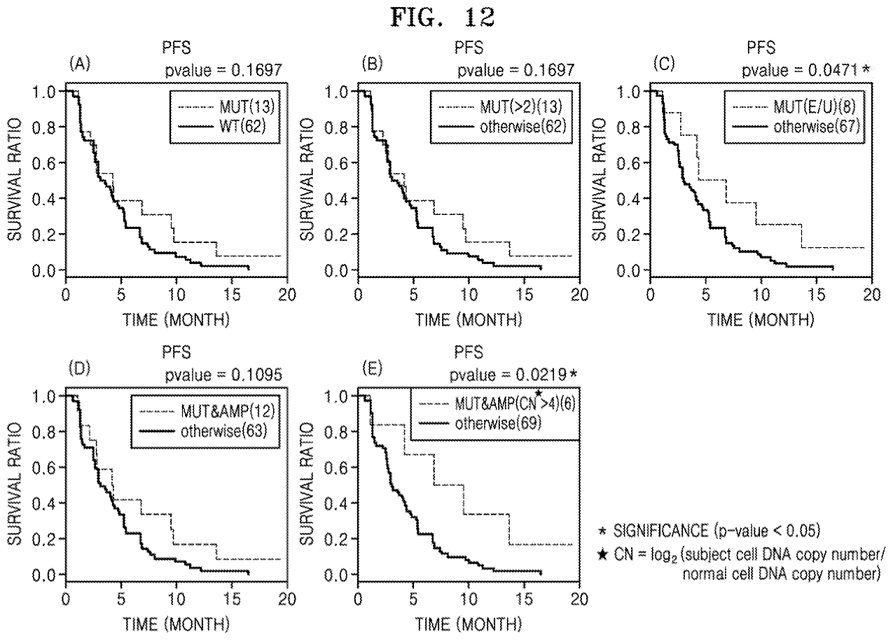

FIG. 12 illustrates graphs showing the correlation between ERBB2 mutation and PFS.

Referring to FIGS. 11 and 12, (A) shows analysis results when there is an ERBB2 mutation, (B) shows analysis results when there are two or more ERBB2 mutations, (C) shows analysis results when ERBB2 mutation exists in an extracellular domain or within an upstream, (D) shows analysis results when there are an ERBB2 mutation and copy number amplification (log₂(ratio)>2), and (E) shows analysis results when there are ERBB2 mutation and copy number amplification (log₂(ratio)>4). Referring to FIGS. 11, (C), (D), and (E) had significance, and referring to FIGS. 12, (C) and (E) had significance.

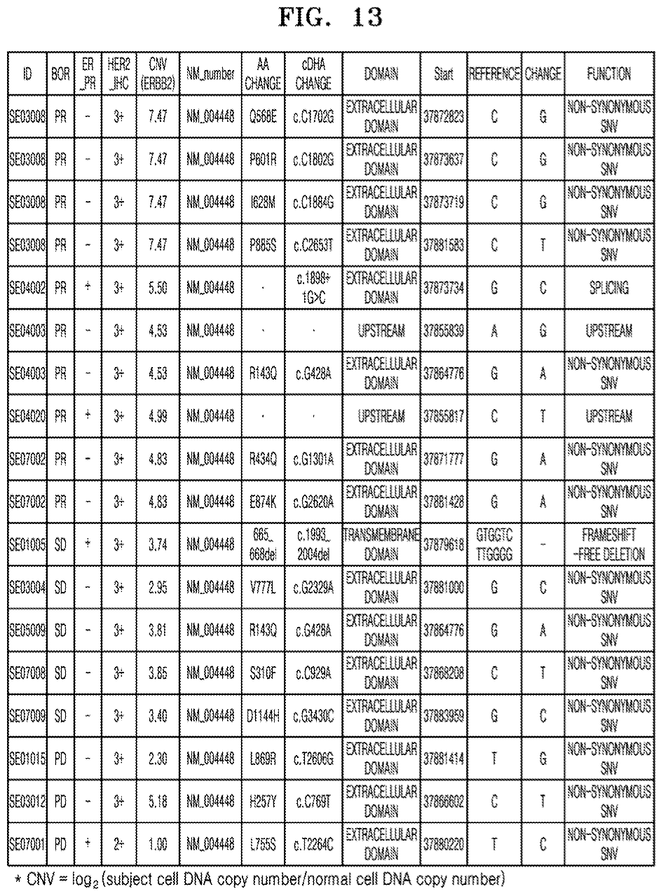

FIG. 13 shows genotype analysis in the ERBB2 gene and upstream region thereof and prognosis following the treatment with poziotinib, regarding 13 patients with mutations from among 75 patients.

Referring to FIGS. 13 to 19, PR represents partial remission, SD represents stable disease, and PD represents progressive disease. EP/PR and IHC indicate whether breast cancer cells have a receptor state in which there are estrogen receptor (ER), progesterone receptor (PR) and HER2 on the surface and cytoplasm and nucleus thereof. EP/PR indicates the state of ER and PR. Immunohistochemistry (IHC) indicates HER2 state of breast cancer cells measured by IHC. All examined cancer cells expressed 2 or more HER2.

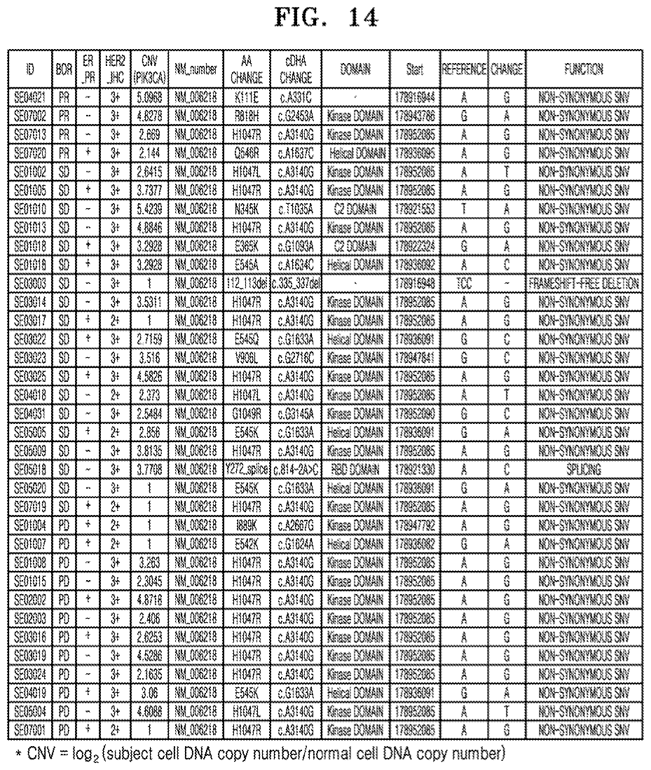

Referring to FIGS. 13 and 14, cells showing therapeutic efficacy when treated with poziotinib all had an ERBB2 gene copy number of which log₂(copy number) was 2 or more, 3 or more, or 4 or more.

AA change and AA position respectively indicate the amino acid at which the mutation occurred and the location thereof. The number indicates the number based on the amino acid sequence of SEQ ID NO: 1. SEQ ID NO: 2 corresponds to the nucleotide sequence of NCBI accession No. NM_004448, and SEQ ID NO: 1 is an amino acid encoded thereby.

Domain represents the domain of ERBB2 where mutation occurred, and ERBB2 includes an extracellular domain (amino acids 23-652), a transmembrane domain (amino acids 653-675), a cytoplasmic domain (amino acids 676-1255), and a protein kinase domain (amino acids 720-987). Upstream represents the mutation that occurred in the upstream of a gene that is not a region encoding ERBB2. Start indicates the number with reference to the nucleotide sequence of chromosome 17.

Referring to FIG. 13, patients 1 to 10 showed partial remission and stable disease which indicates that the poziotinib treatment is effective. The breast cancer may have ERBB2 gene log₂(ratio) of 2 or more, 4 or more, 6 or more, or 16 or more. The breast cancer may be a metastatic HER2-positive breast cancer. In the breast cancer, HER2 may be expressed at a level of 3+ or higher when measured by IHC. The breast cancer may have 2 or more, or 4 or more mutations.

FIG. 14 shows genotype analysis in the PIK3CA gene and prognosis following the treatment with poziotinib, regarding 34 patients with mutations from among 75 patients.

Referring to FIGS. 11 to 14, the copy number represents log₂(ratio).

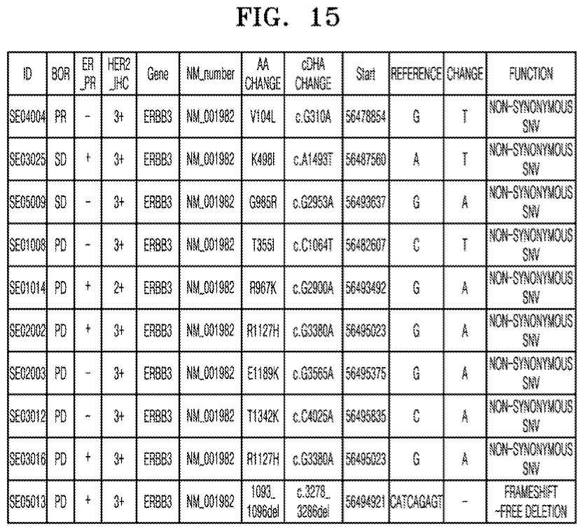

FIG. 15 shows genotype analysis in the ERBB3 gene and prognosis following the treatment with poziotinib, regarding 10 patients with mutations from among 75 patients.

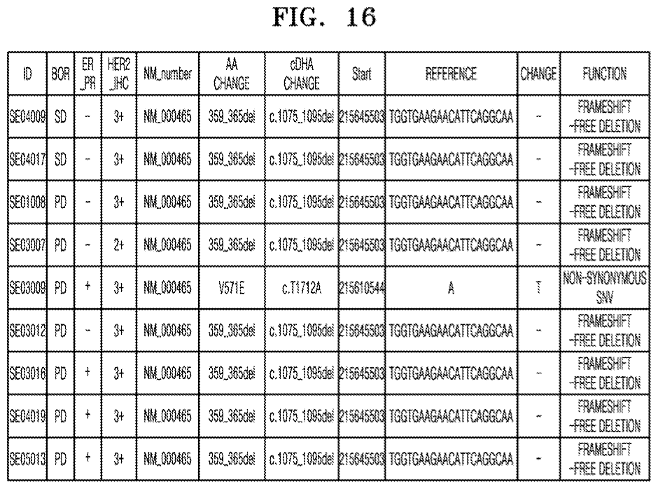

FIG. 16 shows genotype analysis in the BARD1 gene and prognosis following the treatment with poziotinib, regarding 9 patients with mutations from among 75 patients.

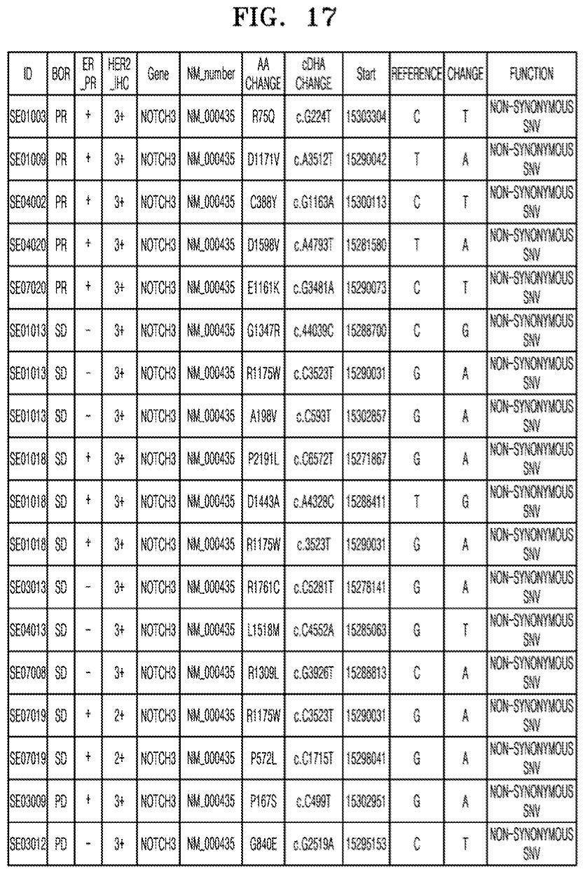

FIG. 17 shows genotype analysis in the NOTCH3 gene and prognosis following the treatment with poziotinib, regarding 12 patients with mutations from among 75 patients.

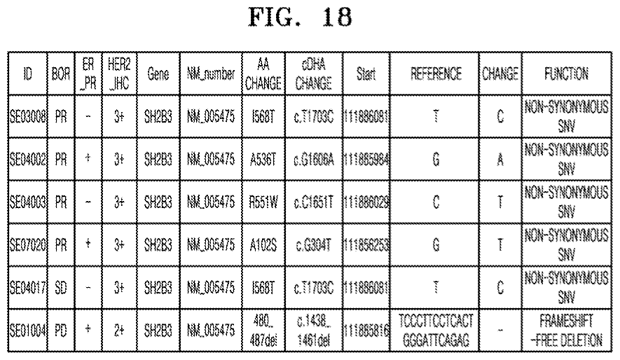

FIG. 18 shows genotype analysis in the SH2B3 gene and prognosis following the treatment with poziotinib, regarding 6 patients with mutations from among 75 patients.

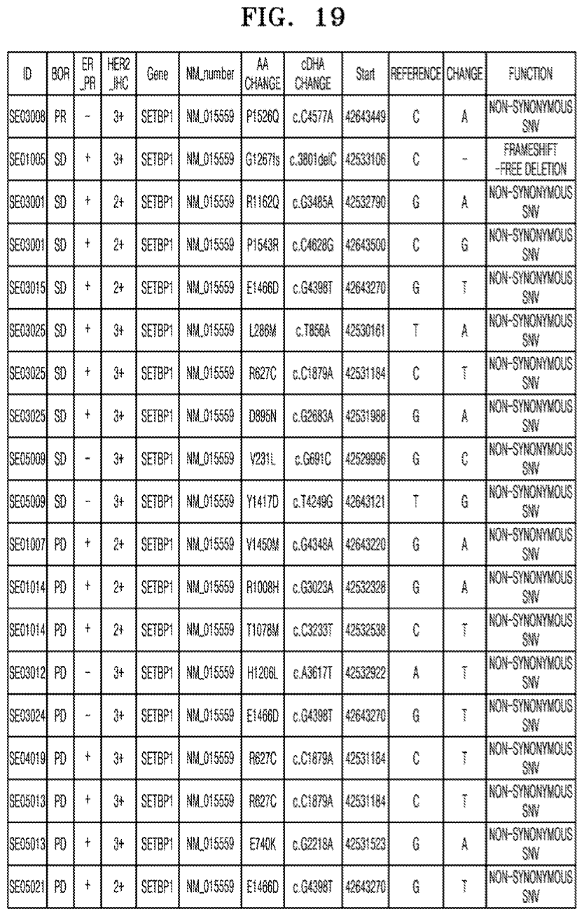

FIG. 19 shows genotype analysis in the SETBP1 gene and prognosis following the treatment with poziotinib, regarding 13 patients with mutations from among 75 patients.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

1. A pharmaceutical composition for the treatment of cancer of a subject, the pharmaceutical composition comprising poziotinib, wherein the subject has cancer cells that have
at least one selected from copy number amplification of the ERBB2 gene, one or more mutations in an ERBB2 gene region including the ERBB2 gene and a sequence within 10 kb upstream of the ERBB2 gene, an ERBB3 wild-type gene, a BARD1 wild-type gene, a SETBP1 wild-type gene, a PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

2. The pharmaceutical composition of claim 1, wherein the cancer comprises breast cancer, ovarian cancer, head and neck cancer, lung cancer, gastric cancer, colon cancer, kidney cancer, blood cancer, or pancreatic cancer.

3. The pharmaceutical composition of claim 1, wherein the subject has a HER2-positive metastatic breast cancer.

4. The pharmaceutical composition of claim 1, wherein the subject has undergone HER2-targeted cancer treatment, not treatment with poziotinib.

5. The pharmaceutical composition of claim 1, wherein the cancer cells have one or more mutations in an extracellular domain-encoding region of the ERBB2 gene or within 10 kb upstream of the ERBB2 gene.

6. The pharmaceutical composition of claim 1, wherein the cancer cells have a log₂(ratio) value of the ERBB2 gene which is 1 or more, 2 or more, or 4 or more, wherein the ratio is obtained by dividing a copy number of the ERRB2 gene in the cancer cells by a copy number of the ERRB2 gene in normal cells.

7. The pharmaceutical composition of claim 1, wherein the cancer cells are sensitive to poziotinib.

8. The pharmaceutical composition of claim 1, wherein a mutation in the ERBB2 gene region includes a nucleotide mutation that causes substitution of at least one amino acid selected from Q568E, P601R, I628M, P885S, R143Q, R434Q, and E874K in the amino acid sequence of ERBB2 of SEQ ID NO: 1, and at least one substitution mutation selected from substitution of G at position 1898 with C in a nucleotide sequence encoding ERBB2 of SEQ ID NO: 2, substitution of A at position 100 with Gin a nucleotide sequence of SEQ ID NO: 3, and substitution of C at position 100 with Tin a nucleotide sequence of SEQ ID NO: 4.

9. The pharmaceutical composition of claim 8, wherein the nucleotide mutation that causes substitution of at least one amino acid selected from Q568E, P601R, I628M, P885S, R143Q, R434Q, and E874K comprises at least one substitution selected from substitution of C at position 1702 with G, substitution of C at position 1802 with G, substitution of C at position 1884 with G, substitution of C at position 2653 with T, substitution of G at position 428 with A, substitution of G at position 1301 with A, and substitution of G at position 2620 with A, in the nucleotide sequence of SEQ ID NO: 2.

10. Use of poziotinib in the preparation of medicaments for the treatment of a subject having cancer, wherein the subject has cancer cells that have
at least one selected from copy number amplification of the ERBB2 gene, one or more mutations in an ERBB2 gene region including the ERBB2 gene and a sequence within 10 kb upstream of the ERBB2 gene, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

11. A method of treating cancer in a subject, the method comprising administering a therapeutically effective amount of poziotinib to the subject having cancer, wherein the subject has cancer cells that have
at least one selected from copy number amplification of the ERBB2 gene, one or more mutations in an ERBB2 gene region including the ERBB2 gene and a sequence within 10 kb upstream of the ERBB2 gene, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

12. The method of claim 11, wherein the cancer comprises breast cancer, ovarian cancer, head and neck cancer, lung cancer, gastric cancer, colon cancer, kidney cancer, blood cancer, or pancreatic cancer.

13. The method of claim 11, wherein the subject has a HER2-positive metastatic breast cancer.

14. The method of claim 11, wherein the subject has undergone HER2-targeted cancer treatment, not treatment with poziotinib.

15. The method of claim 11, wherein the cancer cells have one or more mutations in an extracellular domain-encoding region of the ERBB2 gene or the sequence within 10 kb upstream of the ERBB2 gene.

16. The method of claim 11, wherein the cancer cells have an average number of replication units of the ERBB2 gene of 2 or more.

17. The method of claim 11, wherein a mutation in the ERBB2 gene region comprises a nucleotide mutation that causes substitution of at least one amino acid selected from Q568E, P601R, I628M, P885S, R143Q, R434Q, and E874K in the amino acid sequence of ERBB2 of SEQ ID NO: 1, and at least one substitution mutation selected from substitution of G at position 1898 with C in a nucleotide sequence encoding ERBB2 of SEQ ID NO: 2, substitution of A at position 100 with Gin a nucleotide sequence of SEQ ID NO: 3, and substitution of C at position 100 with T in a nucleotide sequence of SEQ ID NO: 4.

18. The method of claim 17, wherein the nucleotide mutation that causes substitution of at least one amino acid selected from Q568E, P601R, I628M, P885S, R143Q, R434Q, and E874K comprises at least one substitution selected from substitution of C at position 1702 with G, substitution of C at position 1802 with G, substitution of C at position 1884 with G, substitution of C at position 2653 with T, substitution of G at position 428 with A, substitution of G at position 1301 with A, and substitution of G at position 2620 with A, in the nucleotide sequence of SEQ ID NO: 2.

19. A method of treating poziotinib-sensitive cancer in a subject, the method comprising: detecting that a cancer cell-containing sample obtained from the subject has
at least one selected from copy number amplification of the ERBB2 gene, one or more mutations in an ERBB2 gene region including the ERBB2 gene and a sequence within 10 kb upstream of the ERBB2 gene, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene,
wherein having at least one selected from copy number amplification of the ERBB2 gene, one or more mutations in the ERBB2 gene region including the ERBB2 gene and a sequence within 10 kb upstream of the ERBB2 gene, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene indicates that the cancer cells are sensitive to poziotinib; and
administering a therapeutically effective amount of poziotinib to the subject having poziotinib-sensitive cancer.

20. A method of identifying a subject having poziotinib-sensitive cancer, the method comprising:
detecting that a cancer cell-containing sample obtained from a subject has at least one selected from copy number amplification of the ERBB2 gene, one or more mutations in an ERBB2 gene region including the ERBB2 gene and a sequence within 10 kb upstream of the ERBB2 gene, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene,
wherein having at least one selected from copy number amplification of the ERBB2 gene, one or more mutations in an ERBB2 gene region including the ERBB2 gene and a sequence within 10 kb upstream of the ERBB2 gene, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene indicates that the cancer cells are sensitive to poziotinib.

21. The method of claim 19, wherein the cancer comprises breast cancer, ovarian cancer, head and neck cancer, lung cancer, gastric cancer, colon cancer, kidney cancer, blood cancer, or pancreatic cancer.

22. The method of claim 19, wherein the detecting includes requesting a test that provides analysis results for determining whether cancer cells isolated from the subject have at least one selected from copy number amplification of the ERBB2 gene, one or more mutations in an ERBB2 gene region including the ERBB2 gene and a sequence within 10 kb upstream of the ERBB2 gene, the ERBB3 wild-type gene, the BARD1 wild-type gene, the SETBP1 wild-type gene, the PIK3CA wild-type gene, one or more mutations in the NOTCH3 gene, one or more mutations in the SH2B3 gene, copy number amplification of the CDK12 gene, copy number deletion of the BRCA1 gene, copy number deletion of the STAT3 gene, and no copy number variation of the FGFR3 gene.

23. The method of claim 19, wherein, in the detecting, the one or more mutations in the ERBB2 gene region are one or more mutations in an extracellular domain-encoding region of the ERBB2 gene or the sequence within 10 kb upstream of the ERBB2 gene of the ERBB2 gene region.

24. The method of claim 19, wherein the detecting comprises performing at least one analysis selected from sequencing, size analysis, primer extension analysis, allele-specific primer extension analysis, allele-specific nucleotide hybridization analysis, a 5′-nuclease degradation assay, an assay using molecular beacons, an assay based on single-stranded conformation polymorphism, hybridization analysis, and oligonucleotide ligation analysis.

25. The method of claim 19, wherein the detecting comprises measuring the average number of replication units by performing at least one analysis selected from a single nucleotide polymorphism (SNP) array, comparative genomic hybridization (CGH), southern blot analysis, fluorescence in situ hybridization (FISH), and silver in situ hybridization (SISH).

26. The method of claim 19, wherein in the detecting, it is determined that the cancer cells are sensitive to poziotinib when the cancer cells have one or more mutations in the ERBB2 gene region and the average number of replication units of the ERBB2 gene is two or more.

27. The method of claim 19, wherein the detecting comprises providing the cancer cell-containing sample obtained from the subject.

28. A method of treating cancer in a subject, the method comprising administering a therapeutically effective amount of therapeutic drugs other than poziotinib to the subject having cancer, wherein the subject has cancer cells that have at least one selected from
the existence of one or more mutations in the ERBB3 gene, the existence of one or more mutations in the BARD1 gene, the absence of one or more mutations in the NOTCH3 gene, the absence of one or more mutations in the SH2B3 gene, the existence of one or more mutations in the SETBP1 gene, the existence of one or more mutations in the PIK3CA gene, the absence of amplification of the CDK12 gene, the absence of copy number variation of the ERBB2 gene, the absence of copy number variation of the BRCA1 gene, the absence of copy number variation of the STAT3 gene, and the existence of copy number variation of the FGFR3 gene.

29. A method of treating cancer in a subject, the method comprising:
selecting a subject for cancer treatment with breast cancer therapeutic drugs other than poziotinib based on whether cancer cells isolated from the subject have at least one selected from the existence of one or more mutations in the ERBB3 gene, the existence of one or more mutations in the BARD1 gene, the absence of one or more mutations in the NOTCH3 gene, the absence of one or more mutations in the SH2B3 gene, the existence of one or more mutations in the SETBP1 gene, the existence of one or more mutations in the PIK3CA gene, the absence of amplification of the CDK12 gene, the absence of copy number variation of the ERBB2 gene, the absence of copy number variation of the BRCA1 gene, the absence of copy number variation of the STAT3 gene, and the existence of copy number variation of the FGFR3 gene, wherein the having of the at least one indicates that the cancer cells are resistant to poziotinib; and
administering a therapeutically effective amount of the breast cancer therapeutic drugs other than poziotinib to a selected subject.

30. A method of treating poziotinib-resistant cancer in a subject, the method comprising:
detecting that cancer cells obtained from a subject have at least one selected from the existence of one or more mutations in the ERBB3 gene, the existence of one or more mutations in the BARD1 gene, the absence of one or more mutations in the NOTCH3 gene, the absence of one or more mutations in the SH2B3 gene, the existence of one or more mutations in the SETBP1 gene, the existence of one or more mutations in the PIK3CA gene, the absence of amplification of the CDK12 gene, the absence of copy number variation of the ERBB2 gene, the absence of copy number variation of the BRCA1 gene, the absence of copy number variation of the STAT3 gene, and the existence of copy number variation of the FGFR3 gene, wherein the having of the at least one indicates that the cancer cells are resistant to poziotinib; and
administering a therapeutically effective amount of the breast cancer therapeutic drugs other than poziotinib to a subject.

31. A method of identifying a subject having poziotinib-resistant cancer, the method comprising:
detecting that a cancer cell-containing sample obtained from a subject has at least one selected from the existence of one or more mutations in the ERBB3 gene, the existence of one or more mutations in the BARD1 gene, the absence of one or more mutations in the NOTCH3 gene, the absence of one or more mutations in the SH2B3 gene, the existence of one or more mutations in the SETBP1 gene, the existence of one or more mutations in the PIK3CA gene, the absence of amplification of the CDK12 gene, the absence of copy number variation of the ERBB2 gene, the absence of copy number variation of the BRCA1 gene, the absence of copy number variation of the STAT3 gene, and the existence of copy number variation of the FGFR3 gene, wherein having of the at least one indicates that the cancer cells are resistant to poziotinib.

32. The method of claim 28, wherein the cancer comprises breast cancer, ovarian cancer, head and neck cancer, lung cancer, gastric cancer, colon cancer, kidney cancer, blood cancer, or pancreatic cancer.
